(19) United States
(12) Patent Application Publication
(10) Pub. No.: US 2015/0336919 A1
(43) Pub. Date: Nov. 26, 2015

(54) Sesquiterpene lactone-based pharmaceutical composition for treating gastrointestinal diseases

(71) Applicant: CHO DANG PHARM. CO., LTD., Guro-gu Seoul (KR)

(72) Inventors: Ki Woon Kim, Seoul (KR); Hye Dong Yoo, Seoul (KR)

(21) Appl. No.: 14/440,570

(22) PCT Filed: Oct. 4, 2013
(86) PCT No.: PCT/KR2013/008852

§ 371 Date: May 4, 2015

(30) Foreign Application Priority Data
Nov. 5, 2012 (KR) 10-2012-0124017

(51) Int. Cl. C07D 307/93

(52) U.S. Cl. CPC C07D 307/93

(57) ABSTRACT

The present invention relates to a sesquiterpene lactone-based pharmaceutical composition for treating gastrointestinal diseases. More particularly, the present invention relates to a pharmaceutical composition, containing as an active ingredient a sesquiterpene lactone-based derivative derived from parthenolide, for preventing or treating gastritis or gastric ulcers.

### TECHNICAL FIELD

The present invention relates to a pharmaceutical composition containing sesquiterpene lactone derivative as active ingredient for treating the gastrointestinal diseases. More particularly, the present invention relates to a pharmaceutical composition containing sesquiterpene lactone derivative derived from parthenolide as active ingredient for treating or preventing the gastrointestinal diseases, such as, the gastritis and/or stomach ulcer.

### DESCRIPTION OF PRIOR ART

It has been reported that sesquiterpene lactone compounds, such as, epi-tulipinolide, parthenolide, costunolide, tulipinolide, liferolide and/or dihydrochrysanolide are included in the extract from the bark of Liriodendron tulipifera (please refer to R W Doskotch, S L Keely, C D Hufford, F S El-Feraly, Phytochemistry, 14:769-773(1975); R W Doskotch, J H Wilton, F M Harraz, E H Fairchild, C-T Huang, F S El-Feraly, J. Nat. Prod, 46:923-929 (1983)).

Especially, parthenolide has been known as useful active ingredient in the extract of the bark of Liriodendron tulipifera. Therefore, parthenolide has been used for treating the disease. Further, it has been known that parthenolide can modulate the inflammatory disease mediated by NF-κB in vitro, and that it also can induce the apoptosis of acute myeloid leukemia (please refer to M W Feltenstein et al, Pharmacology Biochemistry and Behavior 79 (2): 299-302 (2004)). Further, the inhibition of NF-κB activity can be useful for treating obesity derived from lipopolysaccharide (please refer to S J Zunino et al, Cancer letters 254 (1): 119-127 (2007)).

Therefore, we can anticipate that parthenolide derivative from the extract of the bark of Liriodendron tulipifera may be useful for treating inflammatory disease mediated by NF-κB. Based upon such proposition, the inventors have prepared a number of parthenolide derivatives having various kinds of substitution radicals to select useful compound for treating or preventing the gastritis and/or stomach ulcer. Further, the efficacy for treating or preventing the gastritis and/or stomach ulcer has been measured using these compounds.

Because sesquiterpene lactone compounds are lipid soluble, the low solubility of sesquiterpene lactone compounds occurs in the body fluid, which can cause the insufficient bioavailability.

Even though costunolide and/or tulipinolide extracted from the bark of Liriodendron tulipifera has been useful for treating the gastritis and/or stomach ulcer, these compounds showed the cytotoxicity, especially chromosomal toxicity, which causes the critical struggle for developing it as the medicine.

Therefore, the inventors of present application have synthesized a number of compounds suitable for treating the gastritis and/or stomach ulcer without showing cytotoxicity, especially chromosomal toxicity. The efficacy and safety of these compounds as a drug for treating the gastritis and/or stomach ulcer have been measured through animal test.

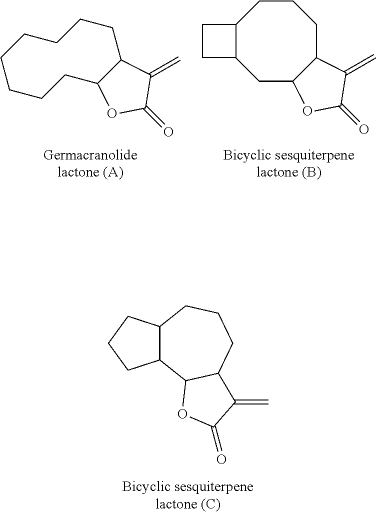

Accordingly, the inventors of present application have prepared bicyclic sesquiterpene lactone compound (B) or bicyclic sesquiterpene lactone compound (C) by cyclizing 10 member ring structure (A) of sesquiterpene lactone, such as, epi-tulipinolide or parthenolide. Further, the inventors of present application have also found that such bicyclic sesquiterpene lactone compound (B) and/or bicyclic sesquiterpene lactone compound (C) show high efficacy as a drug for treating or preventing the gastritis and/or stomach ulcer, while these compounds show very low cytotoxicity, especially chromosomal toxicity.

### Problem to be Solved

The problem to be solved is to develop the bicyclic sesquiterpene lactone compound (B) or the bicyclic sesquiterpene lactone compound (C) by cyclizing 10 member ring structure (A) of sesquiterpene lactone, such as, epi-tulipinolide or parthenolide. Accordingly, it has been found that such bicyclic sesquiterpene lactone compound (B) and/or bicyclic sesquiterpene lactone compound (C) show high efficacy as a drug for treating or preventing the gastritis and/or stomach ulcer, while these compounds show very low cytotoxicity, especially chromosomal toxicity.

### Means for Solving the Problem

The object of present application is to provide a pharmaceutical composition containing sesquiterpene lactone compound represented by formula CD-102 derived from parthenolide for treating or preventing the gastritis and/or stomach ulcer.

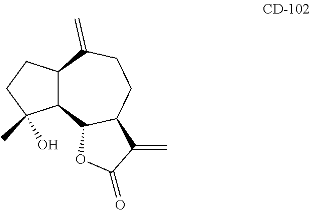

Further, the ED₅₀ of CD-102 compound shows 1.31±0.1 mg/kg according to HCl-ethanol inducing stomach ulcer model.

Further, the ED₅₀ of CD-102 compound shows 1.82±0.1 mg/kg according to indomethacin inducing stomach ulcer model.

Further, said CD-102 compound shows neither cytotoxicity nor chromosomal aberration according to chromosomal aberration test using Chinese hamster lung cell.

The other object of present application is to provide a process for preparing sesquiterpene lactone compound represented by formula CD-102 comprising the steps of:

(1) obtaining the reaction mixture after reacting parthenolide and p-tosylic acid in the presence of methanol solvent, and neutralizing said reaction mixture with disodium phosphate (Na₂HPO₄);

(2) obtaining ethyl acetate layer after separating aqueous layer and ethyl acetate layer by adding ethyl acetate, and drying the solvent in ethyl acetate layer; and

(3) isolating and purifying CD-102 compound using column chromatography and HPLC after dissolving dried product in step (2) with eluent.

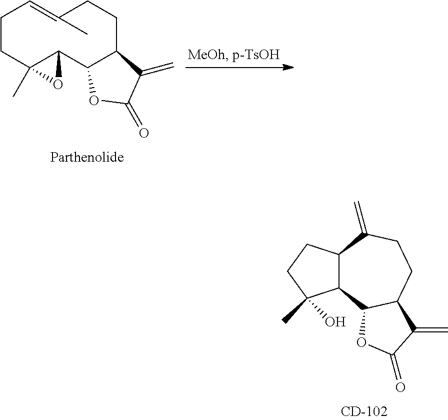

Further, the eluent for column chromatography is mixture of hexane:ethyl acetate (v/v 3:1), while said eluent for HPLC is mixture of water and methanol.

### Advantageous Effect

The outstanding advantageous effect of present application is to afford the bicyclic sesquiterpene lactone compound (B) or the bicyclic sesquiterpene lactone compound (C) by cyclizing 10 member ring structure (A) of sesquiterpene lactone, such as, epi-tulipinolide or parthenolide. Accordingly, such bicyclic sesquiterpene lactone compound (B) and/or bicyclic sesquiterpene lactone compound (C) show high efficacy as a drug for treating or preventing the gastritis and/or stomach ulcer, while these compounds show very low cytotoxicity, especially chromosomal toxicity.

### BRIEF DESCRIPTION OF DRAWINGS

FIG. 1 shows ¹H NMR spectrum of CD-101 compound of present invention.

FIG. 2 shows 2D-COSY NMR spectrum of CD-101 compound of present invention.

FIG. 3 shows ¹H NMR spectrum of CD-102 compound.

FIG. 4 shows 2D-COSY NMR spectrum of CD-102 compound.

FIG. 5 shows ¹H NMR spectrum of CD-103 compound.

FIG. 6 shows 2D-COSY NMR spectrum of CD-103 compound.

FIG. 7 shows HPLC chromatogram indicating the isolation of CD-101, CD-102 and CD-103.

### PREFERRED EMBODIMENT OF INVENTION

The present invention relates to a water soluble pharmaceutical composition containing sesquiterpene lactone compound derived from parthenolide for treating or preventing the gastritis and/or stomach ulcer as well as minimizing cytotoxicity, especially chromosomal toxicity.

The present invention is to provide a pharmaceutical composition containing sesquiterpene lactone compounds represented by formulas CD-101, CD-102 and CD-103 derived from parthenolide for treating or preventing the gastritis and/or stomach ulcer.

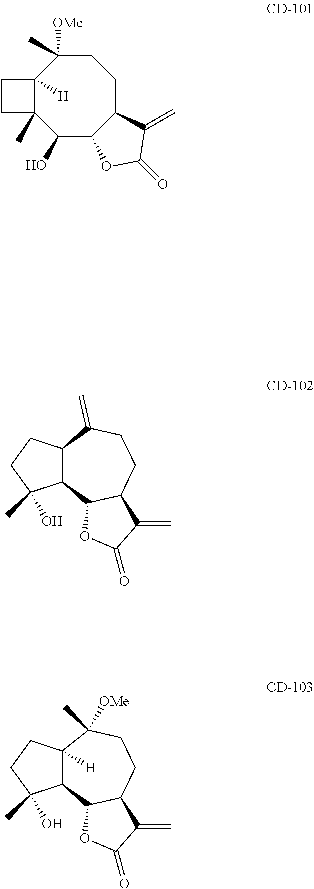

The preparation method of CD-101, CD-102 and CD-103 compounds can be explained as follows.

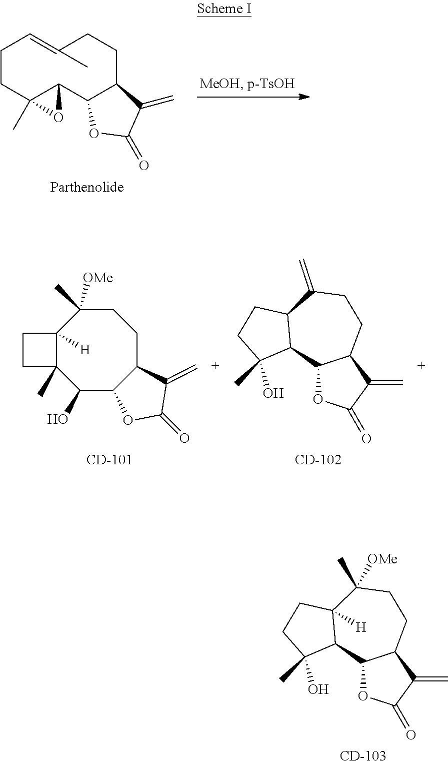

1-1: Synthesis of Compounds

Parthenolide (1000 mg, 4.02 mmol) has been dissolved in methanol (40 mL). Adding p-TsOH (1.529 mg, 8.04 mmol), the mixture has been agitated at room temperature for 24 hours. After neutralizing the reaction mixture with saturated Na₂HPO₄, the water layer and the ethyl acetate layer have been separated by adding ethyl acetate(10 mL 3 times). After adding MgSO₄ to the ethyl acetate layer, the solvent has been dried. The obtained dried sample has been dissolved in mixed solvent of Hexane:EA (v/v 3:1) for column chromatography. After isolating the compounds using HPLC, CD-101 (60 mg, 10%), CD-102 (100 mg, 20%) and CD-103 (30 mg, 5%) have been obtained.

1-2: Isolation of Compounds

For isolation of single component, the component has been dissolved in a concentration of 300 mg/ml with methanol. The aliquot has been collected at 27° C. using HPLC (Agilent 1200series, U.S.A.) having C18 column (Luna 250 10.00 mm 5 um 100 Å, U.S.A.). The mobile phase gradient condition of HPLC can be varied as follows. At 0 minute of retention time, the weight ratio of water and methanol is 40:60 (v/v). Further, the weight ratio becomes 10:90 (v/v) until 5 minutes of retention time, the same weight ratio maintains until 10 minutes, the 100% of methanol becomes at 13 minutes, and same 100% of methanol maintains until 15 minutes.

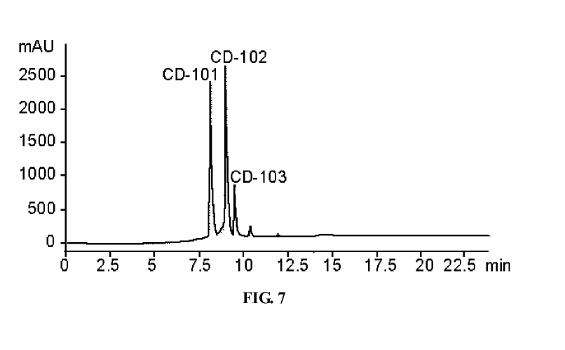

Based upon said mobile phase gradient condition, CD-101 compound has been collected at RT 8.16 minutes. Further, CD-102 compound has been collected at RT 8.99 minutes, while CD-103 compound has been collected at RT 9.53 minutes. The HPLC data of isolated compounds have been shown in FIG. 7. The amount of sample is 30 μl, the flow rate is 3 ml/min and wavelength of UV is 213 nm.

The structural analysis of CD-101 compound using 400 MHz Bruker NMR:

¹H NMR (CDCl₃): 6.25 (1H, d, J=3.25 Hz), 5.58 (1H, d, J=2.78 Hz), 4.19 (1H, dd, J=9.74, 3.25 Hz), 3.59 (1H, d, J=9.79 Hz), 3.17 (3H, s), 3.15-3.12 (1H, m), 2.53 (1H, dd, J=11.09, 8.25 Hz), 2.32˜2.08 (2H, m), 1.93˜1.37 (6H, m), 1.29 (3H, s), 1.25 (3H, s);

MS (LCQ Fleet Ion Trap LC-MS): m/z [M+H]⁺=281

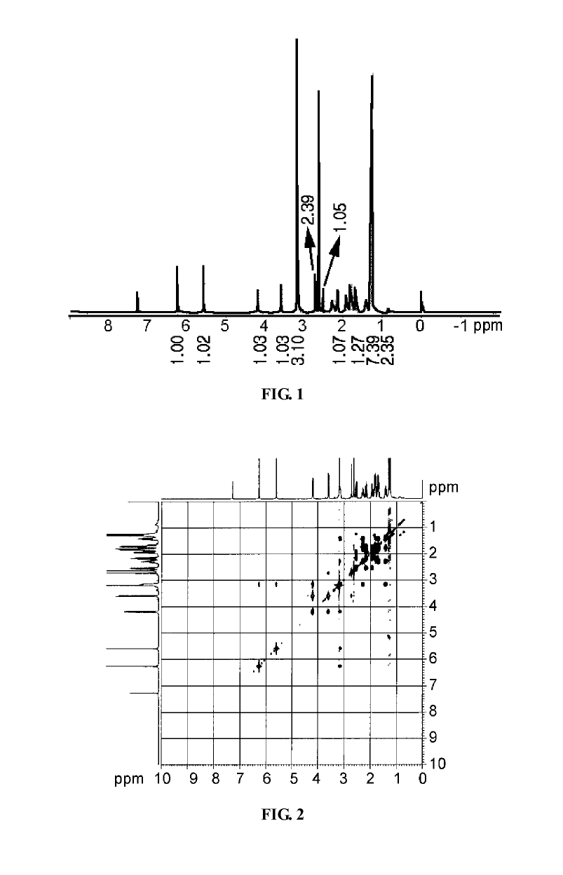

¹H NMR spectrum of CD-101 compound has been shown in FIG. 1. Further, 2D-COSY NMR spectrum of CD-101 compound has been shown in FIG. 2.

The structural analysis of CD-102 compound using 400 MHz Bruker NMR:

¹H NMR (CDCl₃): 6.23 (1H, d, J=3.39 Hz), 5.22 (1H, d, J=3.38 Hz), 4.99 (2H, d, J=14.19 Hz), 4.06 (1H, dd, J=11.51, 9.12 Hz), 2.99 (1H, m), 2.79˜2.66 (2H, m), 2.29˜2.23 (1H, m), 1.98˜1.70 (6H, m), 1.43˜1.33 (1H, m), 1.32 (3H, s);

MS (LCQ Fleet Ion Trap LC-MS): m/z [M+H]⁺=249

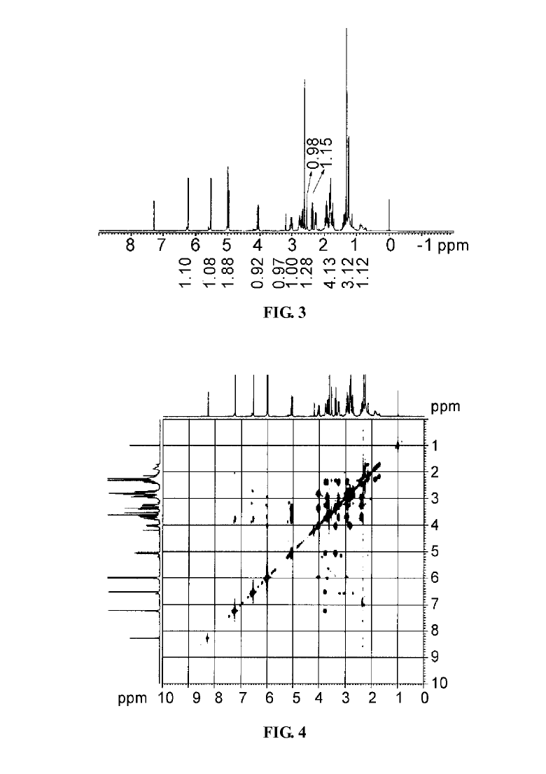

¹H NMR spectrum of CD-102 compound has been shown in FIG. 3. Further, 2D-COSY NMR spectrum of CD-102 compound has been shown in FIG. 4.

The structural analysis of CD-103 compound using 400 MHz Bruker NMR:

¹H NMR (CDCl₃): 6.22 (1H, d, J=3.39 Hz), 5.50 (1H, d, J=3.15 Hz), 4.21 (1H, dd, J=11.63, 9.79 Hz), 3.20 (3H, s), 2.88˜2.74 (2H, m), 2.21˜2.13 (1H, m), 2.05˜1.40 (8H, m), 1.38 (3H, s), 1.16 (3H, s);

MS (LCQ Fleet Ion Trap LC-MS): m/z [M+H]⁺=281

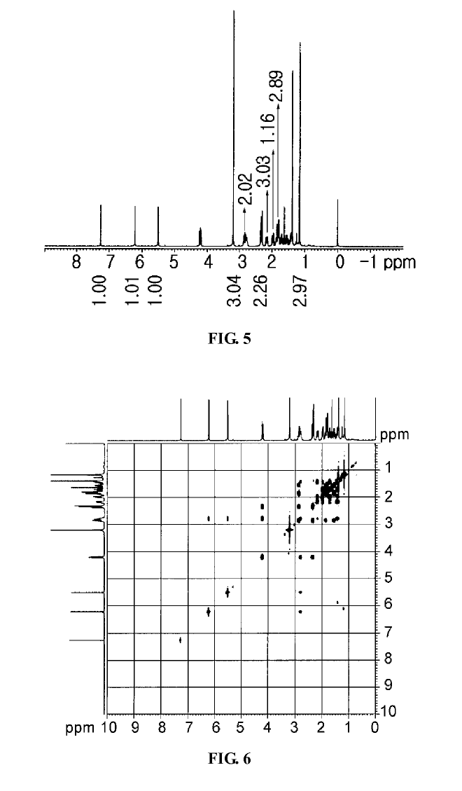

¹H NMR spectrum of CD-103 compound has been shown in FIG. 5. Further, 2D-COSY NMR spectrum of CD-103 compound has been shown in FIG. 6.

The efficacy and safety of obtained compounds have been measured by following Examples.

### Example 1

### Anti-Gastritis Efficacy of CD-101, CD-102 and CD-103 Compound According to HCl-Ethanol Inducing Gastritis Model

After white rats have been fasted for 24 hours, CD-101, CD-102 and CD-103 compound have been orally administered according to method disclosed in Mizui et al., Jap. J. Pharmacol. 33: pp. 939-945 (1983). After 1 hour, 1.5 ml of 60% ethanol including 150 mM HCl has been orally administered to each animal. 1 hour later, animals have been sacrificed by cervical dislocation. After removing the stomach, the stomach has been fixed by injection of 13 ml of 2% formalin for 1 hour. After fixed stomach has been spread out, the lesion area has been measured using the photo by digital camera. Computer program Image J 1.38 (NIH, Bethesda, Md.) has been applied for measuring gastric lesion area (mm²) The lesion inhibition rate has been calculated according to following equation. Further, 50% effective dose (ED₅₀) has been also calculated using regression analysis.

Lesion inhibition rate(%)={gastric lesion area of control group(mm²)−gastric lesion area of drug administration group(mm²)}/gastric lesion area of control group(mm²)×100

Anti-gastritis efficacy of CD-101, CD-102 and CD-103 compound according to HCl-EtOH inducing gastritis model has been shown in Table 1.

The gastric lesion area of control group has been 234.2±30.8 mm² Further, lesion inhibition rate of Rebamipide 50 mg/kg administration group has showed 30%.

Regarding CD-101 administration group, all administration groups of 1, 3, 10 mg/kg have showed anti-gastritis efficacy in the dose dependent manner. In case of 10 mg/kg administration group, 91.8% of lesion inhibition rate has been shown. Further, it has been confirmed that 50% effective dose (ED₅₀) of CD-101 compound is 1.24 mg/kg.

Regarding CD-102 administration group, all administration groups of 1, 3, 10 mg/kg have showed anti-gastritis efficacy in the dose dependent manner. In case of 10 mg/kg administration group, 94.9% of lesion inhibition rate has been shown. Further, it has been confirmed that 50% effective dose (ED₅₀) of CD-102 compound is 1.31 mg/kg.

Regarding CD-103 administration group, all administration groups of 3, 10, 30 mg/kg have showed anti-gastritis efficacy in the dose dependent manner. In case of 30 mg/kg administration group, 99.5% of lesion inhibition rate has been shown. Further, it has been confirmed that 50% effective dose (ED₅₀) of CD-103 compound is 2.55 mg/kg.

If methylene radical is modified, the bicyclic ring compound, either open 10-member ring germacranolide lactone skeleton or 10-member ring germacranolide lactone skeleton, has no anti-gastritis efficacy according to HCl-Ethanol inducing gastritis model (Table 2).

TABLE 1
Efficacy of CD-101, CD-102 and CD-103 compound according to HCl-
Ethanol inducing gastritis model

| | | Number of | Gastric lesion | Lesion inhibition |
|---|---|---|---|---|
| Test group | Dose(mg/kg) | animals | area(mm²) | rate(%) |
| control | — | 5 | 234.2 ± 30.8 | — |
| Rebamipide | 50 | 5 | 163.9 ± 15.6 | 30.0 |
| CD-101 | 1 | 5 | 135.1 ± 43.1 | 42.3 |
| | 3 | 5 | 59.3 ± 23.5* | 74.7 |
| | 10 | 5 | 19.2 ± 11.7* | 91.8 |
| CD-102 | 1 | 5 | 133.5 ± 21.0* | 43.0 |
| | 3 | 5 | 69.4 ± 18.4* | 70.4 |
| | 10 | 5 | 11.9 ± 10.0* | 94.9 |
| control | — | 5 | 187.5 ± 27.0 | — |
| CD-103 | 1 | 5 | 80.8 ± 19.4* | 56.9 |
| | 3 | 5 | 62.1 ± 32.2* | 66.9 |
| | 10 | 5 | 1.0 ± 0.4* | 99.5 |

*: Significant difference compared to the control group (p < 0.05)

TABLE 2
The ineffective compounds according to HCl-Ethanol inducing gastritis
model

| Test | Lesion inhibition | Test | Lesion inhibition |
|---|---|---|---|
| material | rate | compound | rate |
| 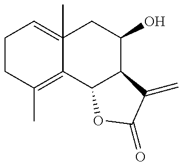 | no effect | 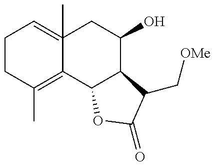 | no effect |
| 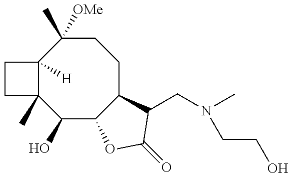 | no effect | 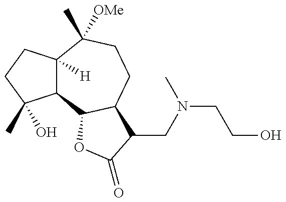 | no effect |
| 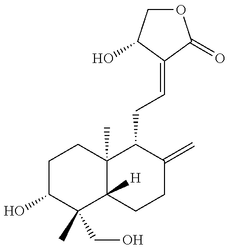 | no effect | 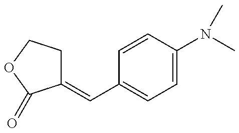 [SMILES: CN(C)c1ccc(/C=C2\CCOC2=O)cc1] | no effect |
| 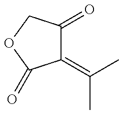 | no effect | 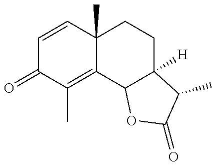 [SMILES: CC1=C2C3OC(=O)[C@@H](C)[C@@H]3CC[C@@]2(C)C=CC1=O] | no effect |

### Example 2

### Anti-Stomach Ulcer Efficacy of CD-101, CD-102 and CD-103 Compound According to Indomethacin Inducing Stomach Ulcer Model

After white rats have been fasted for 24 hours, CD-101, CD-102 and CD-103 compound have been orally administered according to method disclosed in Yamasaki et al., Jap. J. Pharmacol. 49: pp. 441-448 (1989). After 1 hour, indomethacin (50 mg/kg) has been orally administered to each animal After fasting for 5 hours, animals have been sacrificed by cervical dislocation. After removing the stomach, the stomach has been fixed by injection of 13 ml of 2% formalin for 1 hour. After fixed stomach has been spread out, the lesion area has been measured using the photo by digital camera. Computer program Image J 1.38 (NIH, Bethesda, Md.) has been applied for measuring gastric lesion area (mm²) The lesion inhibition rate and 50% effective dose (ED₅₀) have been calculated as the same manner of HCl-EtOH model.

Anti-stomach ulcer efficacy of CD-101, CD-102 and CD-103 compound according to indomethacin inducing stomach ulcer model has been shown in Table 3. The gastric lesion area of control group has been 59.9±8.7 mm².

Regarding CD-101 administration group, all administration groups of 1, 3, 10 mg/kg have showed anti-stomach ulcer efficacy in the dose dependent manner. In case of 10 mg/kg administration group, 73.1% of lesion inhibition rate has been shown. Further, it has been confirmed that 50% effective dose (ED₅₀) of CD-101 compound is 2.78 mg/kg.

Regarding CD-102 administration group, all administration groups of 1, 3, 10 mg/kg have showed anti-stomach ulcer efficacy in the dose dependent manner. In case of 10 mg/kg administration group, 77.2% of lesion inhibition rate has been shown. Further, it has been confirmed that 50% effective dose (ED₅₀) of CD-102 compound is 1.82 mg/kg.

Regarding CD-103 administration group, all administration groups of 1, 3, 10 mg/kg have showed anti-stomach ulcer efficacy in the dose dependent manner. In case of 10 mg/kg administration group, 74.0% of lesion inhibition rate has been shown. Further, it has been confirmed that 50% effective dose (ED₅₀) of CD-103 compound is 2.06 mg/kg.

TABLE 3
Efficacy of CD-101, CD-102 and CD-103 compound according to
indomethacin inducing stomach ulcer model

| | | Number of | Gastric lesion | Lesion inhibition |
|---|---|---|---|---|
| Test group | Dose(mg/kg) | animals | area(mm²) | rate(%) |
| control | — | 7 | 59.9 ± 8.7 | — |
| Rebamipide | 50 | 7 | 30.2 ± 6.4* | 49.6 |
| CD-101 | 1 | 7 | 43.8 ± 7.7 | 26.9 |
| | 3 | 7 | 26.0 ± 4.7* | 56.6 |
| | 10 | 7 | 16.1 ± 4.8* | 73.1 |
| CD-102 | 1 | 7 | 35.0 ± 5.2 | 41.6 |
| | 3 | 7 | 26.3 ± 6.8* | 56.1 |
| | 10 | 7 | 13.6 ± 2.9* | 77.2 |
| CD-103 | 1 | 7 | 37.2 ± 7.0 | 37.9 |
| | 3 | 7 | 25.6 ± 4.7* | 57.3 |
| | 10 | 7 | 15.6 ± 3.1* | 74.0 |

*: Significant difference compared to the control group (p < 0.05)

### Example 3

### Chromosomal Aberration Test Induced by CD-101, CD-102 and CD-103 Compound

For measuring the induction of chromosomal aberration, the chromosomal aberration test has been carried out using Chinese hamster lung (CHL) cell. 2 kinds of methods have been adopted, such as, metabolic activation method (+S9) using metabolic activation system and direct method (−S9) without metabolic activation system.

1: Determination of Test Material Reacting Concentration

For determining the test material reacting concentration, the cell growth inhibition test has been carried out. The test material has been dissolved and diluted in dimethyl sulfoxide (DMSO). Regardless of metabolic activation system, 12 concentration steps (2.43, 4.87, 9.75, 19.5, 39.1, 78.1, 156.3, 312.5, 625, 1250, 2500, 5000 μg/ml) of test material have been prepared. The cells have been cultured for 24 hours as the same way of main test. In case of using metabolic activation system, the medium has been replaced by normal medium after 6 hour from the treatment of metabolic activator. The medium has been further cultured for 18 hours.

Based upon the data of cell growth inhibition test, maximum concentration has been determined when the cell growth has been inhibited up to 50% as the relative cell numbers. The Table 4 shows the concentration of this test.

2: Main Test

Regardless of metabolic activation system, the test materials have been prepared after 24 hours from the initiation of test. Then, the number of chromosomal aberration has been counted. In case of direct method without metabolic activation system, the frequency of chromosomal aberration has not meaningfully increased compared to negative control in all reacting concentration. Further, in case of using metabolic activation system, the frequency of chromosomal aberration also has not meaningfully increased compared to negative control in all reacting concentration, according to the treatment of metabolic activator for 6 hours as well as the recovery of treated material for 18 hours (Table 4).

It has been confirmed that CD-101, CD-102 and CD-103 compound do not induce the chromosomal aberration as to the CHL cell. However, it has been also confirmed that open 10-member ring germacranolide lactone derivatives induce the chromosomal aberration as to CL cell. From this result, we can find out that 10-member ring germacranolide lactone structure has been changed into bicyclic ring structure not to induce the chromosomal aberration (Table 5).

TABLE 4

| | Metabolic | | |
|---|---|---|---|
| Test material | Activation System | Dose(μg/kg) | Results |
| negative control | +S9 | 0 | negative |
| 1% DMSO | −S9 | 0 | negative |
| Positive control | +S9 | 20 | positive |
| Benzo(a)pyrene | −S9 | 0.5 | positive |
| Mitomycin C | | | |
| CD-101 | +S9 | 40 | negative |
| | | 20 | negative |
| | | 10 | negative |
| | | 10 | negative |
| | −S9 | 5 | negative |
| | | 2.5 | negative |
| CD-102 | +S9 | 20 | negative |
| | | 10 | negative |
| | | 5 | negative |
| | | 5 | negative |
| | −S9 | 2.5 | negative |
| | | 1.25 | negative |
| CD-103 | +S9 | 10 | negative |
| | | 5 | negative |
| | | 2.5 | negative |
| | | 2.5 | negative |
| | −S9 | 1.25 | negative |
| | | 0.625 | negative |

TABLE 5
Chromosomal aberration test

| | Metabolic | |
|---|---|---|
| | activation | |
| Test material | system | Result |
| negative control | +S9 | negative |
| (1% DMSO) | −S9 | negative |
| 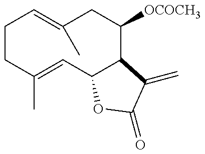 | +S9 −S9 | positive positive |
| 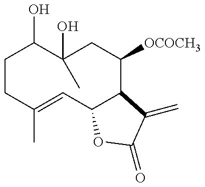 | +S9 −S9 | positive positive |
| 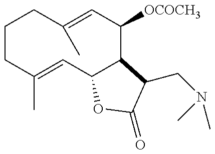 | +S9 −S9 | positive positive |
| 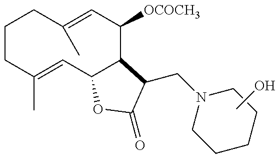 | +S9 −S9 | positive positive |

What is claimed is:

1. A pharmaceutical composition containing sesquiterpene lactone compound represented by formula CD-102 derived from parthenolide for treating or preventing the gastritis and/or stomach ulcer.

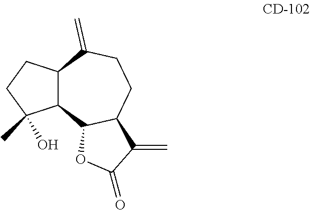

2. The pharmaceutical composition according to claim 1, the ED₅₀ of CD-102 compound shows 1.31±0.1 mg/kg according to HCl-ethanol inducing stomach ulcer model.

3. The pharmaceutical composition according to claim 1, the ED₅₀ of CD-102 compound shows 1.82±0.1 mg/kg according to indomethacin inducing stomach ulcer model.

4. The pharmaceutical composition according to claim 1, the CD-102 compound shows neither cytotoxicity nor chromosomal aberration according to chromosomal aberration test using Chinese hamster lung cell.

5. A process for preparing sesquiterpene lactone compound represented by formula CD-102 comprising the steps of:
(1) obtaining the reaction mixture after reacting parthenolide and p-tosylic acid in the presence of methanol solvent, and neutralizing said reaction mixture with disodium phosphate (Na₂HPO₄);
(2) obtaining ethyl acetate layer after separating aqueous layer and ethyl acetate layer by adding ethyl acetate, and drying the solvent in ethyl acetate layer; and
(3) isolating and purifying CD-102 compound using column chromatography and HPLC after dissolving dried product in step (2) with eluent.

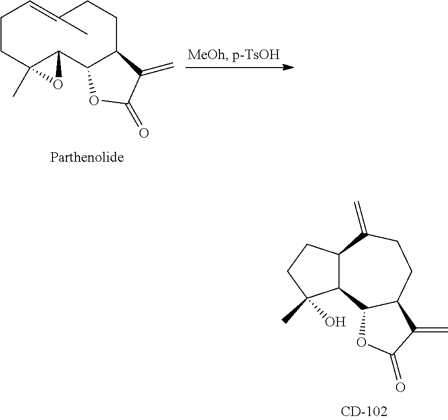

6. The process according to claim 5, the eluent for column chromatography is mixture of hexane:ethyl acetate (v/v 3:1), while the eluent for HPLC is mixture of water and methanol.
